(19) United States
(12) Patent Application Publication
(10) Pub. No.: US 2021/0202046 A1
(43) Pub. Date: Jul. 1, 2021

(54) PREDICTIVE DETERMINATION OF EXCESS CHEMICAL POTENTIALS

(71) Applicant: ROQUETTE FRERES, Lestrem (FR)

(72) Inventors: Oumar TOURE, BETHUNE (FR); Baptiste BOIT, LA GORGUE (FR); Claude-Gilles DUSSAP, CLERMONT FERRAND (FR)

(21) Appl. No.: 17/057,337

(22) PCT Filed: May 28, 2019
(86) PCT No.: PCT/FR2019/051259

§ 371 Date: Nov. 20, 2020

(30) Foreign Application Priority Data
May 28, 2018 (FR) 18 54525

(51) Int. Cl. G16C 20/30

(52) U.S. Cl. CPC G16C 20/30

(57) ABSTRACT

Method for the predictive determination of an excess chemical potential including the following steps:
a. performing a typology of the chemical species which are present in a solution, in order to determine at least one true species,
b. associating a vector of physical magnitudes with each true species determined in step a
c. performing an operation on at least one of said vectors of physical values in order to determine an excess chemical potential of an apparent species.

The invention relates to the field of the predictive determination of the physicochemical properties of solutions.

The predictive determination of the physicochemical properties of a solution is of great industrial value. Specifically, since an industrial facility is required to process large volumes, it is important to control as best as possible the various parameters of a manufacturing process which uses a solution, in order to ensure good matching between the facility and the calibration of the process. For example, some facilities will not be able to process volumes that are too large, others will only be able to process liquids and it will thus be necessary to ensure that no boiling will occur, and yet others will be able to process gases but only within a given pressure range above which safety problems will arise. Furthermore, it may be valuable to precisely know the amount of thermal energy to be supplied to a product needed for its curing or processing, in order to avoid other interfering reactions and excessive energy consumption.

Determination of the parameters and calibration of industrial facilities is thus of major importance. Although it is sometimes possible to perform this calibration by trial and error, this method is not always satisfactory as it cannot guarantee that the operating conditions are optimal. Consequently, the use of predictive determination methods, for example performed using simulation software, are particularly valuable. These predictive determination methods make it possible to model activity coefficients or chemical potentials of molecules.

In this respect, it is known from WO 2015/175387 and WO 2012/051242 to model the activity coefficient of a molecule in a solution of interest by approaching each molecule using an empirical algorithm for averaging the charge density profile (or molecular histogram of charge densities), similar to the method described in the publication by Klamt et al., J. Phys. A, 1998, Volume 102, No. 26, pages 5074-5085, this method being known as the “sigma-averaging method”. These models use a purely physical and combinatory approach, which may be satisfactory in the case of certain simple and dilute aqueous solutions, but gives far from realistic results when attempting to model concentrated solutions within which chemical equilibria exist, notably equilibria of chemical associations (complexation, solvation, hydration, etc.) deduced from the binding properties between the molecules making up the solution. Specifically, these models cannot reflect the chemical interactions between the different molecules. This is notably the case for concentrated aqueous solutions containing sugars and/or polyols and/or carbohydrates.

Now, in an industrial context, the components of interest within a solution are generally the solutes and not the solvent, and the purely physical and combinatory approach is rarely suited to these concentrated aqueous solutions.

There is therefore a need for a method for the predictive determination of physicochemical magnitudes of equilibria in an industrial context, in particular in the case of aqueous solutions which are concentrated or which have a very high solids content. Specifically, attempts at modeling aqueous solutions have already been made. In this regard, Catté et al. (Fluid Phase Equilibria 105, 1-25, 1995) combined the UNIFAC physical model with a chemical model of fixed hydration of sugars by water, in order to model aqueous solutions which are rather dilute. However, the Applicant has observed that this type of model does not work with concentrated aqueous solutions.

The invention succeeds in doing this via the use of a method for the predictive determination of an excess chemical potential of an apparent species, involving the following steps:
a. performing a typology of the chemical species which are present in a solution, in order to determine at least one true species,
b. associating a vector of physical magnitudes with each true species determined in step a,
c. performing an operation on at least one of said vectors of physical magnitudes in order to determine an excess chemical potential of an apparent species in the solution.

The term “true species” means a chemical entity which is present in a solution and which cannot always be isolated. For example, in the case of an aqueous sorbitol solution, the isolated sorbitol, the sorbitol associated with one water molecule, the sorbitol associated with two water molecules and the sorbitol associated with three water molecules, will be distinguished as that number of true species.

The term “apparent species” means the solubilized species which can be isolated, the properties of which can readily be measured. For example, in the case of an aqueous solution of sorbitol, this is water, on the one hand, and sorbitol, on the other hand.

The notions of “true species” and “apparent species” are known per se. For example, Prausnitz et al. has already described them in the reference text Molecular thermodynamics of fluid-phase equilibria, Prentice-Hall international series in the physical and chemical engineering sciences, Prentice-Hall PTR., 1999, page 353. They also appear in various publications such as those from Toure et al. (The Canadian Journal of Chemical Engineering 93, 2015, page 445 and also in Fluid Phase Equilibria, 2016, 424, page 92). Achard et al., (AIChE journal, 40(7), 1994. 1210-1222) and the abovementioned publication by Catté et al. also refer to the respective properties of true species when they mention “true concentration or mole fraction”, and of apparent species when they mention “apparent concentration or mole fraction”. Alternatively, a true species may also be referred to as a molecular entity and an apparent species may also be referred to as a chemical species (or set of molecular entities) such as defined by the IUPAC.

The excess chemical potential is determined from a partial molar property, which leads to the chemical potentials after comparison between various solutions.

Preferably, the method for the predictive determination of an excess chemical potential according to the invention also involves a step consisting in performing a paving in order to approach each true species determined in the step a with a combination of Platonic solids, so that it is associated with a form having an outer surface consisting of a combination of flat surface segments of the same area.

The determination of an excess chemical potential, also called the excess free enthalpy, is particularly valuable as it makes it possible to determine most of the physicochemical properties of the solution. Specifically, the excess chemical potential of the solution contributes notably toward reflecting the availability of the molecules.

The term “chemical species” is not intended to be limited to only one given molecule. The same molecule may correspond to several different chemical species depending on whether or not it is interacting with one or more other molecules. By virtue of the fact that the typology is done on the basis of chemical species, and not on the basis of molecules, the step consisting in performing a typology of the chemical species which are present can reflect the interactions within the solution. During this step, the probability of the presence of a given species can also be taken into account, in order to reflect various physicochemical equilibria. In other words, the “molecule” is an “apparent species” present in the mixture and the “chemical species” is a “true species” actually present in the mixture. For example, the molecule could be sorbitol and the corresponding chemical species would be the various hydration states of sorbitol.

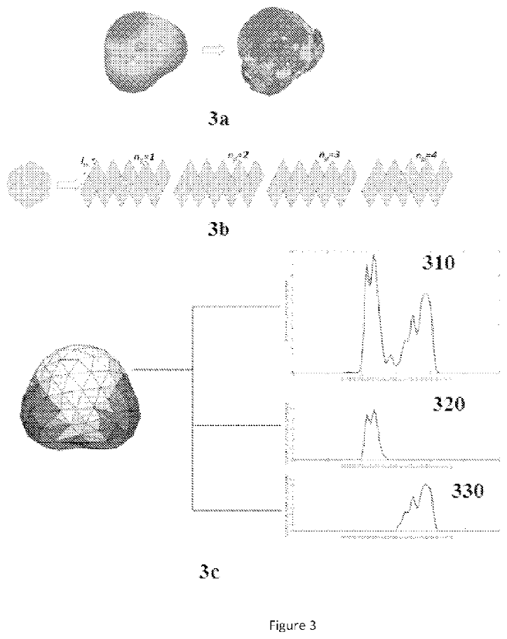

After determining the typology of the various true species present, it is necessary to generate a certain amount of input data beforehand. These input data are drawn from the result of a quantum calculation, for example in the form of a COSMO file for each chemical species. An example of visualization of a COSMO file content is illustrated in FIG. 3a, where the color represents the content value of the physical magnitude vector associated with a true species. As a reminder, a COSMO file comprises, for each of the segments forming the outer surface of the chemical species, the electrostatic potential, the area, the charge and the surface charge density.

Preferably, and in particular when the physical magnitude vector comprises surface charge densities, each chemical species identified is then approached by a combination of identical Platonic solids. A Platonic solid is a regular and convex polyhedron having outer surfaces which are identical on each of its faces, which allows it to pave a three-dimensional space without discontinuities.

The use of identical Platonic solids is thus a major innovation. Specifically, once the quantum calculation is completed, the segments forming the outer surface of a molecule have various sizes; the usual methods for modeling the activity coefficient (notably those of the models mentioned above) consist in empirically averaging the surface charge densities of said segments by an identical flat surface (generally square or circular) of a suitable size. Through the use of identical Platonic solids, all the space representing the volume of the solution is paved in a compact manner and each molecule has an outer surface consisting of a combination of identical surfaces, of genuine surface segments, which makes it possible to compare the molecules with each other and to model the interactions between different segments in order to be able to combine, in a coherent manner, a physical model with a chemical model. This notably allows the modeling of the physicochemical properties of the abovementioned concentrated aqueous solutions.

To this end, at least one physical magnitude vector is associated with each determined true species. If a Platonic solids paving has been used, preferably one physical magnitude vector is generated per surface segment. The term “vector” is to be understood herein as its algebraic meaning: it is a table collating a number of physical magnitudes relative to the surface segment with which it is associated.

When each vector is connected to a surface segment of a similar size, each vector has the same weight.

The vectors as a whole are thus a good description of the solution, from which it is possible to predict an excess chemical potential of an apparent species, which is particularly useful in the context of an industrial facility as this enables control of the quality of matching of the predictive method with the reality, by means of a measurement.

The determination of the respective excess chemical potentials of the true species is performed using a physical view of the interactions between molecules and/or between chemical species. An astute choice of the typology of the true species thus enables the association, in a coherent manner, of this physical view of the interactions with a chemical model of chemical associations, and thus recomposition of the solution with equivalent species in order to construct the prediction of the respective excess chemical potentials of the apparent species.

The solution is preferably an aqueous solution of sugars and/or of polyols and/or of carbohydrates. Specifically, the model according to the invention is particularly suited to solutions of this type which can be very concentrated and contain a great number of different chemical species, notably due to the numerous water molecules which can be adsorbed by the molecules.

Carbohydrates include sugars and polyols. The terms “sugars”, “polyols” and “carbohydrates” are well known to those skilled in the art. Polyols are also known by the term “alditols”. For each of these terms, a person skilled in the art may refer to the definitions of the International Union of Pure and Applied Chemistry (IUPAC) quoted in the article by Moss et al., Pure Applied Chemistry, 1995, 67, 1307, Glossary of class names of organic compounds and reactivity intermediates based on structure (IUPAC Recommendations 1995).

Carbohydrate solutions may notably be obtained from starch. As for aqueous sugar solutions, these may be a solution of maltodextrins, a glucose syrup or a fructose syrup.

As examples of sugars, mention may be made of glucose, arabinose, xylose, fructose, psicose (also called allulose), mannose, ribose, galactose, trehalose, cellobiose, gentiobiose, isomaltose, isomaltulose, kojibiose, laminaribiose, maltose, galactose, lactose, maltulose, nigerose, saccharose and sophorose.

Carbohydrates other than the abovementioned sugars may be saccharides with a degree of polymerization of greater than or equal to 3. They may be oligosaccharides with a degree of polymerization of greater than or equal to 3, notably oligosaccharides with a degree of polymerization of from 3 to 20, and notably oligosaccharides such as maltotriose, isomaltotriose, panose, raffinose, maltotetratose and cyclodextrins. Generally, the polyols may be any abovementioned saccharide which is hydrogenated. They may be, for example, polyglucitol, i.e. a solution of glucose syrup or of hydrogenated maltodextrin. The polyols may be polyols comprising from 3 to 24 carbon atoms, for instance glycerol, erythritol, threitol, arabitol, xylitol, ribitol, mannitol, sorbitol, galactitol, fucitol, iditol, inositol, volemitol, isomalt, maltitol, lactitol, maltotriitol and maltotetraitol. Preferably, the polyols are chosen from sorbitol, mannitol, xylitol and maltitol.

The combination of Platonic solids is, for example, a combination of icosahedra. An icosahedron has twenty triangular faces. It is the type de Platonic solid which has the greatest number of faces and thus makes it possible to obtain a paving best approaching each chemical species present. Alternatively, the Platonic solid is a dodecahedron made of twelve faces of identical regular pentagons.

Preferably, at least one of said physical magnitude vectors contains a physical magnitude chosen from the charge density, the tendency to form hydrogen bonding as a hydrogen atom donor, the tendency to form hydrogen bonding as a hydrogen atom acceptor, and the electrostatic potential. Instead of the charge density, it is of course also possible to use the charge and/or the area as the physical magnitude.

These physical values make it possible to describe as best as possible the interactions in the solution and to derive the properties thereof, and in particular the relevant different chemical potentials, in a precise manner. According to the physical magnitudes used, the equations used to get back to the chemical potential will not be the same.

According to the type of solution used, the interactions between chemical species may be different and it may thus be valuable to use other, more appropriate, physical magnitudes.

The typology of the chemical species present can, for example, distinguish various complexation states of the same molecule.

Preferably, the typology of the chemical species which are present enables distinction between various solvation states, in particular hydration states, of the same molecule. It is understood that solvation is a particular mode of complexation with solvent, hydration being solvation with water. This typology of chemical species thus makes it possible to associate the abovementioned physical view of the determination of the excess chemical potentials of the chemical species, with a contribution of chemical associations deduced from the bonding properties between the molecules making up the solution.

Therefore, the chemical species truly present in the mixture have physical properties which are different from those of the individual molecules forming the apparent mixture. Consequently, recomposition of the solution with equivalent species makes it possible to construct the prediction of the partial molar properties leading to the determination of the excess chemical potential of the molecules (or apparent species).

The method for the predictive determination of an excess chemical potential according to the invention may also include a step c′ consisting in producing a molecular histogram of at least one of said physical magnitudes.

Such a histogram enables a statistical treatment of the solution. A sigma-profile is an example of such a histogram, applied to the charge density profile of a solution.

The method for the predictive determination of an excess chemical potential according to the invention may also include a step consisting in determining a magnitude deduced from the excess chemical potential of the molecules (or apparent species). Knowing the excess chemical potential makes it possible to reflect the shift from ideality of solutions and to deduce all the thermodynamic properties (water activity a_{w}, activity coefficients γi of the constituents, osmotic coefficient) and equilibria between phases (liquid-liquid, liquid-vapor and liquid-solid equilibria), from the general relationships of thermodynamics. For example, the liquid-vapor equilibrium is characterized by the notion of the boiling point of the mixture. Preferably, this magnitude deduced from the chemical potential is chosen from an activity coefficient of a chemical species or of a molecule present, a boiling point of the solution, and a solubility of a chemical species or of a molecule present.

Specifically, the excess chemical potential of the solution makes it possible to find a large number of magnitudes of interest of the solution, which may serve the purpose of a particular facility made to follow the method according to the invention.

The solution preferably has a mass content of solvent of less than 30% relative to the total mass of the solution, advantageously less than 20%.

Specifically, although the predictive determination method according to the invention is suited to any type of solution, the accuracy of the results obtained is particularly valuable in the field of concentrated solutions for which there is no simple alternative.

The chosen Platonic solids have, for example, a characteristic size of between 0.0005 Å and 100 Å, preferably between 0.0005 Å and 6 Å. The characteristic size of a Platonic solid corresponds to the size of its edge. A characteristic size in this range allows a sufficiently accurate rendering of the properties of the solution.

A subject of the invention is also the use of a method according to the invention for calibrating an industrial facility. Specifically, any type of industrial facility requires calibration. In view of the volumes involved in an industrial facility, performing this calibration in a predictive manner, without having to sacrifice part of the production for this purpose, is particularly advantageous.

The industrial facility is preferably a facility for producing sugars and/or polyols and/or carbohydrates. Specifically, as mentioned above, the method according to the invention is particularly suited to solutions of sugars and/or of polyols and/or more generally of carbohydrates.

A subject of the invention is also a computer system comprising a processor, said processor being configured to implement a method according to the invention when it receives specific instructions.

A subject of the invention is also a computer program product comprising a code configured to implement a method according to the invention when it is run by a processor or an electronic control unit.

The invention may be understood more clearly with the aid of the nonlimiting implementation examples described hereinbelow, and on examining the attached drawing, in which:

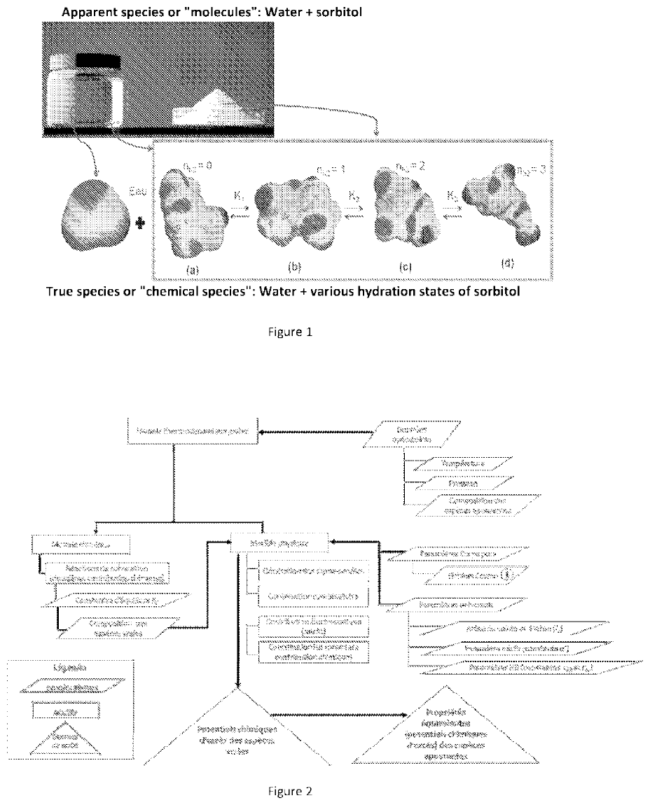

FIG. 1 is a representation of the three-dimensional structure of sorbitol and of its various hydration states,

FIG. 2 is a diagram representing the global structure and the data necessary for the implementation of a method according to the invention,

FIG. 3 represents various steps for the generation of a paving of Platonic solids of a molecule and corresponding charge density histograms,

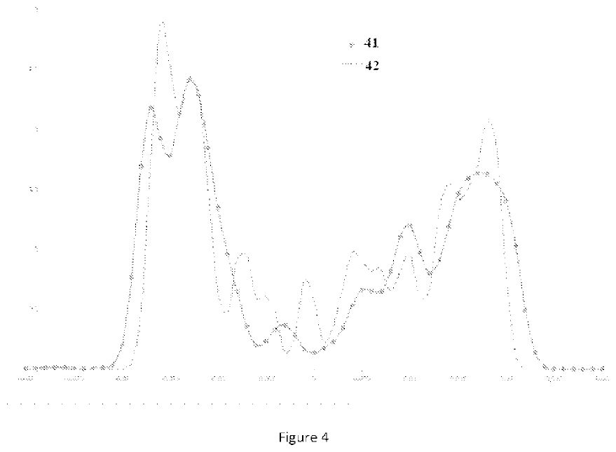

FIG. 4 represents a comparison of molecular histograms of charge densities (σ-profile), regarding the representative case of the water molecule, obtained using the abovementioned method of Klamt et al. and the method of the invention,

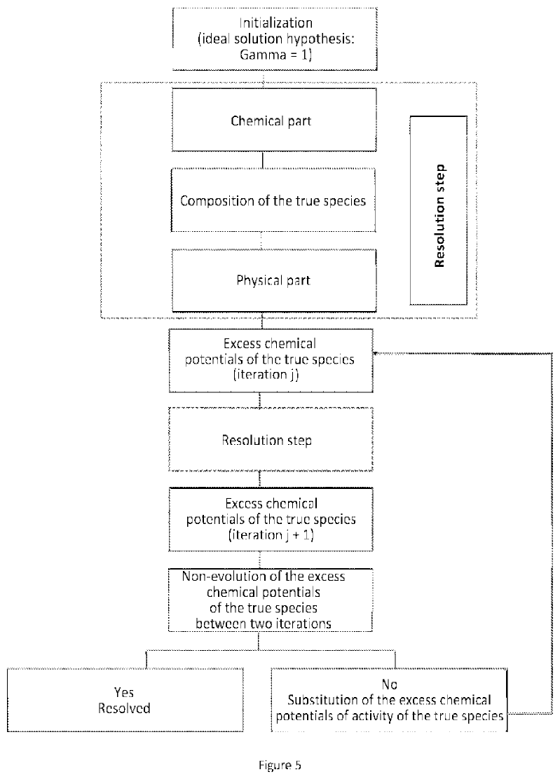

FIG. 5 represents a diagram showing a resolution method enabling the calculation of the various excess chemical potentials of a solution by performing the method according to the invention,

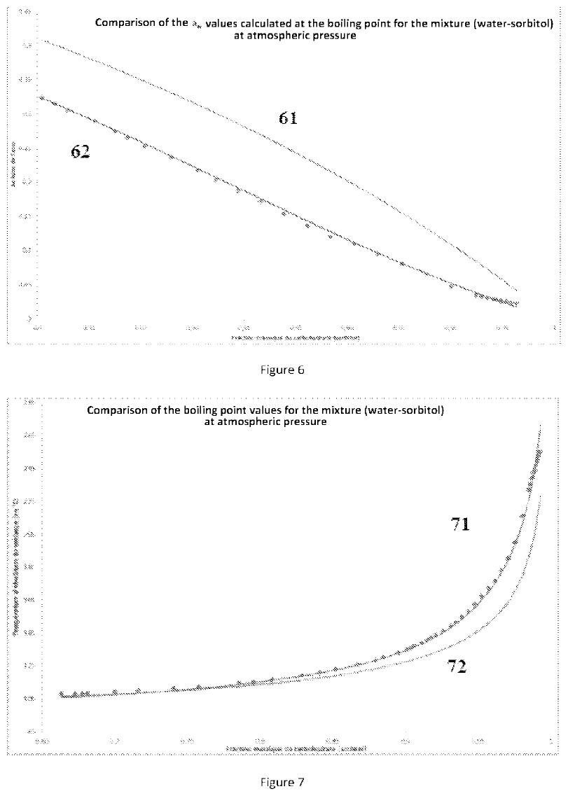

FIG. 6 is a comparison of the water activity data obtained by performing the method according to the invention, using a method according to the prior art, and by calculating them from the boiling points at atmospheric pressure, obtained by experimental measurement,

FIG. 7 is a comparison of the boiling point data obtained using the thermodynamic model developed in the present invention, those obtained using methods from the prior art, and after obtaining them by experimental measurement,

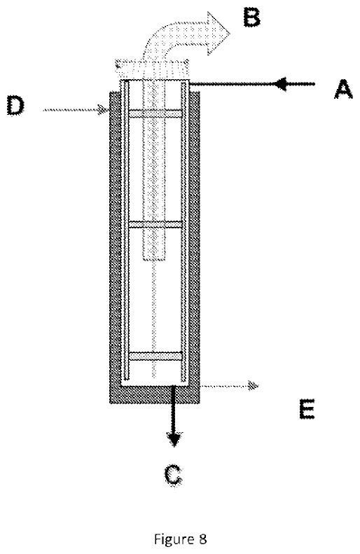

FIG. 8 is a schematic representation of a thin-film scraped-surface evaporator.

The following examples will enable the present invention to be understood more clearly, without, however, limiting the scope thereof.

In order to determine the typology of the various chemical species present, it is necessary to determine the various physicochemical equilibria which may take place within the solution.

In the case of this nonlimiting implementation example, the case of sorbitol is considered. Sorbitol is capable of adsorbing 0, 1, 2 or 3 water molecules according to the amount of water available. Five chemical species may thus be present; these are the species illustrated in FIG. 1.

After determining the typology of the various true species present, it is necessary to generate a certain amount of input data beforehand. These input data are drawn from the result of a quantum calculation, for example in the form of a COSMO file for each chemical species. As a reminder, a COSMO file comprises, for each of the segments forming the outer surface of the chemical species, the area, the charge, the charge density and the electrostatic potential. An example of visualization of a COSMO file content is illustrated in FIG. 3a, where the color represents the content value of the physical magnitude vector associated with a true species.

These COSMO files form part of the input data of the physical model, as illustrated in FIG. 2. Moreover, operating data (temperature, pressure, composition of the various molecules or apparent species) and universal parameters of the physical model are also used to determine the chemical potentials of the true species by using, in the chemical part, the equilibrium constants of chemical associations between the molecules (solvation) in the chemical model. Recomposition of the solution with equivalent species thus makes it possible to determine the excess chemical potentials of the molecules (or apparent species) from knowledge of the true compositions and the respective excess chemical potentials of the chemical species actually present in the solution. To do so, the molecular surface of each chemical species is paved beforehand with a combination of Platonic solids in order to associate it with a form having an outer surface made of a combination of flat surface segments of the same area, and a physical magnitude vector is associated with each surface segment.

A Platonic solid is a regular and convex polyhedron having outer surfaces which are identical on each of its faces.

Preferably, the Platonic solid is a dodecahedron consisting of 12 faces of identical and regular pentagons, most preferentially an icosahedron consisting of 20 identical equilateral triangular faces (FIG. 3b). As these solids have a surface which is closer to that of a sphere, the paving of the surface is closer to the value of the surface derived from the quantum calculation and contained in the COSMO file (FIG. 3a).

Preferentially, the value of the size (lp) of the edge of these Platonic solids is less than or equal to 6 Å. For each chemical species, the molecular sigma-profile, i.e. a histogram giving the charge density profile, 310 (p(σ)) is generated using a paving of the surface with a whole number of Platonic solids and allocating to each face, or surface segment, a value that is stored in a vector.

This generation makes it possible to avoid the use of a sigma-averaging method and thus to stay as close as possible to the quantum simulations.

FIG. 3c illustrates the sigma profile 310 obtained.

For each surface segment, i.e. each face of a Platonic solid, corresponding to a portion of chemical species which is capable of reacting as a hydrogen bonding (HB) donor (ad) of a molecule, donor sigma-profiles 320 (pHB(σd)) are generated, these sigma-profiles being generated using a paving of their surface with a whole number of Platonic solids, this paving being preferentially identical to the preceding one.

Similarly, for each hydrogen bonding (HB) acceptor segment (σa) of a molecule, acceptor sigma-profiles 330 (pHB(σa)) are generated, these sigma-profiles being generated using a paving of their surface with a whole number of Platonic solids, this paving being preferentially identical to the preceding one.

Each surface segment is thus allocated three values in a vector corresponding to each of the profiles 310, 320, and 330.

Said Platonic solids used in the various pavings have identical edges and are thus Platonic solids of identical areas and volumes.

As represented in FIG. 4, the sigma-profile 41 thus obtained is different from the sigma-profile 42 obtained with the methods of the prior art using sigma-averaging. FIG. 4 represents the molecular sigma-profile as a function of the surface charge density in electrons per square Angstrom.

The sigma-profile of the mixture is the sum weighted by the mole fractions of the sigma-profiles of each of the molecules.

The sigma-profile is thus used hereinbelow as the descriptor of the paved chemical species.

In the physical part of the method according to the invention, as illustrated in FIG. 2, after generating the sigma-profiles of the chemical species, the step for obtaining the excess chemical potential of a molecule in a mixture involves the obtention of three different contributions.

The combinatory contribution takes into account the size and shape differences between the molecules present in the mixture and reflects the probability that each face meets another face.

The electrostatic contribution, known as the “contribution misfit”, is generated so as to take into account the fact that the electrostatically interacting surfaces are faces of the Platonic solid.

The “HB” contribution is obtained by taking into account the fact that it only results from interactions between the hydrogen bonding (HB) donor parts and acceptor parts contained in the acceptor and donor sigma-profiles, respectively, of the mixture.

Combinatory Contribution

In order to take into account the size and shape differences between the molecules present in the mixture, the combinatory contribution of the excess chemical potential is calculated using the following formula:

μᵢ^{E,combi}=RT·ln(γᵢ^{Combi}); wherein

$$ln ( {γ}_{i}^{Combi} ) = ln ( \frac{{Φ}_{i}}{{x}_{i}} ) + ( 1 - \frac{{Φ}_{i}}{{x}_{i}} ) + \frac{{q}_{i}}{{r}_{i}} \cdot ln ( \frac{{θ}_{i}}{{Φ}_{i}} ) + \frac{{q}_{i}}{{r}_{i}} \cdot ( \frac{{Φ}_{i}}{{x}_{i}} - \frac{{θ}_{i}}{{x}_{i}} )$$

This formula is novel in the state of the art. It is an adaptation of the Staverman Guggenheim (SG) type formula and makes it possible to take into account an entropic contribution which considers the volume and outer surface differences of the molecules

$${Φ}_{i} = \frac{{x}_{i} \cdot {r}_{i}}{{\sum }_{j = 1}^{{n}_{prat}} {x}_{j} \cdot {r}_{j}}$$

$${θ}_{i} = \frac{{x}_{i} \cdot {q}_{i}}{{\sum }_{j = 1}^{{n}_{prat}} {x}_{j} \cdot {q}_{j}}$$

$${q}_{i} = \frac{{A}_{i}}{{s}_{icosa \dot{e} dre}}$$

$${r}_{i} = \frac{{V}_{i}}{{v}_{icosa \dot{e} dre}}$$

Contribution Misfit

The contribution misfit of the excess chemical potential results from the following formula:

μᵢ^{E,misfit}=μᵢ^{misfit,S}−μᵢ^{misfit,ref}

wherein μᵢ^{misfit,S} refers to the contribution misfit of the chemical potential of the molecule in the solution and μᵢ^{misfit,ref} its chemical potential in a reference state chosen by the user. This reference state may be chosen either from a “pure body” reference state for each of the constituents or from an “infinite dilution” reference state in the main solvent (e.g. water) for each of the constituents, except for the main solvent which is in a “pure body” reference state.

μᵢ^{misfit,S} is the sum, weighted by the number of segments represented in the sigma-profile pᵢ(σ), of the activity coefficients of the surface segments making up the molecule:

μᵢ^{misfit,S}=∫pᵢ(σ)·μ^{misfit,S}(σ)dσ

The chemical potential of the surface segment μ^{misfit,S}(σ) depends on the electrostatic interaction energies between all the surface segments.)

$${μ}^{misfit , s} ( σ ) = - k \cdot T \cdot ln ( \int \overline{{p}^{s} ( {σ}^{′} )} \cdot exp \frac{{μ}^{misfit , s} ( {σ}^{′} ) - {E}_{misfit} ( σ , {σ}^{′} )}{k \cdot T} ) d {σ}^{′}$$

This interaction energy may be calculated using the following formula:

$${E}_{misfit} ( σ , {σ}^{′} ) = {α}^{′} \cdot \frac{{( {e}_{0} \cdot {10}^{20} ( σ + {σ}^{′} ) )}^{2}}{4 \cdot {ɛ}_{0}} \cdot \sqrt{\frac{{( {s}_{p} \cdot {10}^{- 10} )}^{3}}{π}}$$

wherein α′ refers to one of the universal parameters of the physical model, as illustrated in FIG. 2.

HB Contribution

The HB contribution of the excess chemical potential is calculated using the following formula:

μᵢ^{E,HB}μᵢ^{HB,S}−μᵢ^{HB,ref}

μᵢ^{HB,S} being the HB contribution of the chemical potential of the molecule in the solution and

μᵢ^{HB,ref} being its chemical potential in a reference state chosen by the user.

μᵢ^{HB,S} is the sum of the donor and acceptor contributions.

μᵢ^{HB,S}=∫pᵢ^{HB}(σ_{d})·μ^{HB,S}(σ_{d})dσ_{d}+∫pᵢ^{HB}(σₐ)·μ^{HB,S}(σₐ)dσₐ

The first term of this sum, being the HB donor contribution of the chemical potential, is calculated as being the sum, weighted by the number of the HB acceptor segments represented in the HB acceptor sigma-profile, of the chemical potentials of the HB donor surface segments making up the molecule.

The chemical potential of the HB donor surface segment depends on the HB interaction energies between all the donor surface segments and acceptor surface segments.

$${μ}^{HB , S} ( {σ}_{d} ) = - k \cdot T \cdot ln ( \int \overline{{p}^{HB , S} ( {σ}_{a} )} \cdot exp ( \frac{{μ}^{HB , S} ( {σ}_{a} ) - {E}_{HB} ( {σ}_{a} , {σ}_{d} )}{k \cdot T} ) d {σ}_{a} )$$

This interaction energy may be calculated using the following formula:

$${E}_{HB} ( {σ}_{d} , {σ}_{a} ) = {c}_{HB} \cdot \frac{{e}_{0}^{2} \cdot ( {σ}_{d} , {σ}_{a} )}{4 \cdot π \cdot {ɛ}_{0}} \cdot \frac{{s}_{p}^{2}}{{r}_{da} \cdot {10}^{- 10}}$$

r_{da} is the minimum approach distance between the HB bonding donor and acceptor. c_{HB} and r_{da} refer to the universal parameters of the physical model, as illustrated in FIG. 2.

The second term of this sum, being the HB acceptor contribution of the chemical potential, is calculated as being the sum, weighted by the number of the HB donor segments represented in the HB donor sigma-profile, of the chemical potentials of the HB acceptor surface segments making up the molecule.

The chemical potential of the HB acceptor surface segment also depends on the HB interaction energies between all the donor surface segments and the acceptor surface segments.

$${μ}^{HB , S} ( {σ}_{a} ) = - k \cdot T \cdot ln ( \int \overline{{p}^{HB , S} ( {σ}_{d} )} \cdot exp ( \frac{{μ}^{HB , S} ( {σ}_{d} ) - {E}_{HB} ( {σ}_{a} , {σ}_{d} )}{k \cdot T} ) d {σ}_{d} )$$

Both HB donor and HB acceptor contributions are mutually dependent and are thus calculated simultaneously and iteratively.

This formulation of the “hydrogen bonding” contribution is entirely novel. This contribution is, for example, referred to as the “first chemical contribution”.

The resulting model simultaneously takes into account this first chemical contribution, the combinatory contribution and the electrostatic contribution.

When a carbohydrate such as sorbitol is dissolved in water, the carbohydrate becomes hydrated one or more times, thus forming hydrated species i.e. new chemical species containing a carbohydrate molecule strongly bound to one or more water molecules via hydrogen bonding, in particular in the first hydration sphere. This phenomenon is illustrated in FIG. 1, where the typology of the chemical species which are present in a solution of sorbitol and water (apparent species) is determined. The true species thus determined consist of water, anhydrous sorbitol, and sorbitol which is hydrated once, twice and three times. Moreover, the colored surface surrounding each true species makes it possible to pictorially illustrate the content of the physical magnitude vector associated with said true species.

A chemical model is thus necessary to take into account the contribution of chemical associations (solvation, complexation, etc.) deduced from the bonding properties between the molecules making up the solution, such as the interactions of chemical nature between the sorbitol and the solvent, in particular the successive hydration reactions of sorbitol describing the chemical formation of the hydrated forms of the carbohydrate. This contribution is referred to, for example, as the “second chemical contribution”.

These reactions, and thus the composition of the various anhydrous or hydrated true species present in the mixture, are characterized by hydration equilibrium constants, which are themselves dependent on the chemical potential of each of the true species present in the mixture.

A method for the resolution of physicochemical equilibria is thus necessary in order to determine the respective excess chemical potentials of the true species (free solvent, anhydrous species and hydrated species). Such a resolution method is illustrated in FIG. 5.

To this end, generation of the COSMO file done in the first step of the process must be performed for each chemical species, in the sense of each true species, present in the mixture so as to make all the data generated coherent.

This method, which is likened to a chemical model, makes it possible to determine the equilibrium properties of the mixture using the resolution method indicated in FIG. 5 to determine the excess chemical potentials of the true species present in the mixture and to deduce therefrom all the physicochemical properties deduced from the knowledge of this physicochemical reality of the interactions taking place in the mixture.

To this end, as illustrated in FIG. 2, the user simply needs to indicate the operating conditions (or data) such as the apparent composition, the temperature and/or the pressure to use the thermodynamic model developed in this invention in order to predict the chemical potentials of the chemical species, and thus the composition of the true species, which are present in the real mixture and the equilibrium properties of said mixture.

A comparison is made between the water activity values in a concentrated sorbitol solution obtained via a method of the prior art (in this instance according to the UNIFAC group contribution model) and the method of the invention. Curves 61 (prior art) and 62 (invention) of FIG. 6 are respectively obtained, which represent the water activity as a function of the mass fraction of sorbitol in the solution. Experimental data are superposed thereon and it is found that the invention makes it possible to obtain data that are much closer to the measured parameters.

A comparison is made between the boiling point values at atmospheric pressure in a concentrated sorbitol solution obtained via a method of the prior art (in this instance according to the UNIFAC group contribution model) and the method of the invention. Curves 71 (prior art) and 72 (invention) of FIG. 7 are respectively obtained, which represent the boiling point in ° C. as a function of the mass fraction of sorbitol in the solution. Experimental data are superposed thereon and it is found that the invention makes it possible to obtain data that are much closer to the measured parameters.

In the design of final products such as sorbitols marketed in the form of concentrated liquid solutions, knowledge of the control parameters of equipment such as an evaporator is essential. This is likewise the case for the sorbitol powders generally manufactured using a molten sorbitol solution, i.e. a solution having a very high solids content, which may be above 90%, or even 99%.

Evaporation is the step during which dilute solutions are concentrated by transforming the liquid solvent (for example water) into gas. This is one of the most energy-intensive processes in the chemical and agrifood industries, which leads manufacturers of equipment of this type to offer a wide range of technologies and systems to adapt to the product characteristics, to the constraints associated with the dry material and to the energy costs. It is thus particularly advantageous to be able to optimize the process or simulation costs.

### Example: Evaluation of the Scraped-Film Evaporation Technique for the Continuous Production of Molten Sorbitol from an Aqueous Sorbitol Solution

The functioning of a thin-film scraped-surface evaporator as shown in FIG. 8 is simulated. Stream A represents the stream of solution to be evaporated (aqueous sorbitol solution). The heating steam is introduced into the jacket of the evaporator via Stream D. During the evaporation step, the water evaporated from the sorbitol solution is extracted from the evaporator via Stream B. The heating steam and the heating steam condensates are extracted from the jacket via Stream E. The concentrated solution (molten sorbitol) is thus extracted from the evaporator via Stream C.

A thin-film scraped-surface evaporator has a high global exchange coefficient, which makes it possible very efficiently to evaporate very concentrated solutions, which may have a high viscosity.

To perform this simulation in which the apparent species are water and sorbitol, the typology of the true species present is first determined as illustrated in FIG. 1 (water, anhydrous sorbitol, sorbitol hydrated once, sorbitol hydrated twice and sorbitol hydrated three times).

Quantum calculations are performed for each of these true species using the TURBOMOLE software which makes it possible to generate COSMO files associated with each true species.

The surface charge densities used in the physical magnitude vector are drawn from these COSMO files.

Paving of the outer surface of each true species using Platonic solids of icosahedral type having a characteristic size of 1.12 Å is performed.

As illustrated in FIG. 3c for water, for all the surface segments composing the true species, the molecular sigma values 310 (p(σ)) are generated and are stored as values in the physical magnitude vector of the corresponding true species.

Similarly, for all the surface segments composing the true species, the hydrogen bonding (HB) donor sigma-profiles 320 (pHB(σd)) are generated and are stored as values in the physical magnitude vector of the corresponding true species.

Similarly, for all the surface segments composing the true species, the hydrogen bonding (HB) acceptor sigma-profiles 330 (pHB(σa)) are generated and are stored as values in the physical magnitude vector of the corresponding true species.

The physicochemical equilibrium resolution method illustrated in FIG. 5 was inserted into the ProSimPlus software (sold by the company Prosim) to determine the respective excess chemical potentials of the true species (free solvent, anhydrous species and hydrated species).

To simulate the evaporation process, the following operating conditions are also entered into the ProSimPlus software:
apparent species composition of the solution to be evaporated: 70% sorbitol and 30% water;
inlet temperature of the solution to be evaporated: see Table 1;
inlet flow rate of the solution to be evaporated: see Table 1;
absolute pressure in the evaporator: 50 mbar;
apparent species composition of the concentrated solution (molten sorbitol): see Table 1;
exchange area of the evaporator: see Table 1;
global exchange coefficient of the evaporator: 1000 W/m2/° C.

The thermodynamic model developed in this invention is used under these operating conditions to iteratively predict, with the aid of the ProSimPlus software, the excess chemical potentials of the apparent species, after having predicted the excess chemical potentials of the true species present in the real mixture and the equilibrium properties of said mixture.

The boiling point and the outlet flow rate of the mixture leaving the evaporator are thus calculated (see table 1).

TABLE 1

| | | | 1 | 2 | 3 | 4 | 5 | 6 | 7 | 8 |
|---|---|---|---|---|---|---|---|---|---|---|
| | Scale | | Pilot | Pilot | Industrial | Industrial | Industrial | Industrial | Industrial | Industrial |
| Simulation | Exchange area | m² | 0.14 | 0.14 | 3.00 | 6.00 | 6.00 | 6.00 | 6.00 | 6.00 |
| input data | Flow rate of sorbitol solution | kg/h | 25 | 30 | 915 | 1500 | 1500 | 1800 | 1800 | 1800 |
| | feed | | | | | | | | | |
| | Feed temperature | (° C.) | 40 | 40 | 40 | 40 | 60 | 60 | 60 | 60 |
| | Molten sorbitol dry matter | % | 99.25 | 99.25 | 99.25 | 99.25 | 99.25 | 99.25 | 95.00 | 99.50 |
| Simulation | Heating steam temperature | (° C.) | 139.5 | 146.7 | 166.9 | 154.4 | 154.4 | 167.8 | 121.1 | 173.5 |
| results | Heating steam pressure | (bar) | 3.555 | 4.347 | 7.327 | 5.341 | 5.341 | 7.490 | 2.054 | 8.595 |
| | Temperature of concentrated | (° C.) | 123.7 | 123.7 | 123.7 | 123.7 | 123.7 | 123.7 | 73.0 | 130 |
| | product | | | | | | | | | |
| | Heating steam flow rate | Kg/h | 10.0 | 12.8 | 390.0 | 644.9 | 644.9 | 732.9 | 530.8 | 755.7 |
| | Exchanged power | kW | 6.37 | 7.64 | 233.04 | 382.03 | 356.33 | 427.59 | 326.13 | 437.93 |
| | MLTD (Mean logarithmic | (° C.) | 45.5 | 54.6 | 77.7 | 63.7 | 59.4 | 71.3 | 54.4 | 73.0 |
| | temperature difference) of the | | | | | | | | | |
| | evaporator | | | | | | | | | |
| | Amount of water evaporated | kg/h | 7.37 | 8.84 | 269.66 | 442.07 | 442.07 | 530.48 | 473.68 | 533.67 |
| | Molten sorbitol outlet flow rate | kg/h | 17.64 | 21.16 | 645.34 | 1057.93 | 1057.93 | 1269.52 | 1326.32 | 1266.33 |

### CONCLUSION OF THE EXAMPLE

Among the control parameters determined by simulation by means of the process of the invention, the delay in boiling, i.e. the difference between the boiling point of a solution containing sorbitol and that of the pure solvent (water) is one of the most critical items of data.

Furthermore, regulation of the heating steam (flow rate, pressure, temperature) is important: it conditions the regularity and stability of the evaporator.

The use of the invention for dimensioning (determining the design and scale-up) is thus illustrated in Table 1 and calibrating an industrial facility using the scraped-film evaporation technique.

The simulations performed in Table 1 thus make it possible to dimension and pilot a thin-film scraped-surface evaporator as a function of the needs and uses (steam, food product, etc.) by indicating the optimum parameters to approach the point of functioning as rapidly as possible.
These simulations in fact make it possible to limit the number of scale-up and start-up tests of an industrial facility.

It is understood that the embodiments described are not limiting and that it is possible to make improvements to the invention without departing from the scope thereof.

Unless otherwise specified, the term “or” is equivalent to “and/or”. Similarly, the term “a(n)” or “one” is equivalent to “at least one” unless otherwise specified.

1. A method for the predictive determination of an excess chemical potential of an apparent species, comprising the steps of:
a. performing a typology of the chemical species which are present in a solution, in order to determine at least one true species,
b. associating a vector of physical magnitudes with each true species determined in step a, and
c. performing an operation on at least one of said vectors of physical values in order to determine an excess chemical potential of an apparent species.

2. The method according to claim 1, wherein the solution is an aqueous solution of sugars and/or of polyols and/or of carbohydrates.

3. The method according to claim 1, further comprising performing a paving in order to approach each true species determined in step a with a combination of Platonic solids, so that it is associated with a form having an outer surface consisting of a combination of flat surface segments of the same area.

4. The method according to claim 3, wherein the combination of Platonic solids is a combination of icosahedra.

5. The method according to claim 1, wherein at least one of said physical magnitude vectors contains a physical magnitude chosen from the charge density, the tendency to form hydrogen bonding as a hydrogen atom donor, the tendency to form hydrogen bonding as a hydrogen atom acceptor, and the electrostatic potential.

6. The method according to claim 1, wherein the typology of the chemical species present enables distinction between various complexation states of the same molecule.

7. The method according to claim 1, wherein the typology of the chemical species present enables distinction between various solvation states, in particular hydration states, of the same molecule.

8. The method according to claim 1, further comprising step b′ of producing a molecular histogram of at least one of said physical magnitudes.

9. The method according to claim 1, further comprising determining a magnitude deduced from the excess chemical potential, preferably chosen from an activity coefficient of a chemical species or of a molecule present, a boiling point of the solution, and a solubility of a chemical species or of a molecule present.

10. The method according to claim 1, wherein the solution has a solvent mass content of less than 30% relative to the total mass of the solution, advantageously less than 20%.

11. The method according to claim 3, wherein the Platonic solids have a characteristic size of between 0.0005 Å and 100 Å, preferably between 0.0005 Å and 6 Å.

12. A method for the calibration of an industrial facility by predictive determination of an excess chemical potential of an apparent species, comprising the steps pf:
a. performing a typology of the chemical species which are present in a solution, in order to determine at least one true species,
b. associating a vector of physical magnitudes with each true species determined in step a, and
c. performing an operation on at least one of said vectors of physical values in order to determine an excess chemical potential of an apparent species.

13. The method according to claim 12, wherein the industrial facility is a facility for producing sugars and/or polyols and/or carbohydrates.

14. A computer system, comprising a processor configured to implement a method as claimed in claim 1 when it receives specific instructions.

15. A computer program product comprising a code configured to implement a method as claimed in claim 1 when it is run by a processor or an electronic control unit.
